(19) United States
(12) Patent Application Publication
(10) Pub. No.: US 2013/0012795 A1
(43) Pub. Date: Jan. 10, 2013

(54) APPARATUS AND METHODS FOR SENSING A PARAMETER WITH A RESTRAINT DEVICE

(75) Inventors: Stephen P. Moenning, Punta Gorda, FL (US); Anne E. Moenning, Punta Gorda, FL (US); John Acker, Ft. Myers, FL (US)

(73) Assignee: COLLAR ID LLC, Punta Gorda, FL (US)

(21) Appl. No.: 13/540,031

(22) Filed: Jul. 2, 2012

Related U.S. Application Data
(60) Provisional application No. 61/504,343, filed on Jul. 5, 2011.

(51) Int. Cl. A61B 5/1455; A61F 5/37; A61B 5/024; A61B 5/145; A61B 5/01

(52) U.S. Cl. 600/340; 600/309; 600/549; 600/509

(57) ABSTRACT

A restraint device includes at least a first restraint portion defining a space for receiving at least a first body portion of a wearer to restrain movement of the wearer. The restraint device also includes at least one sensor on the first restraint portion and configured to sense at least one parameter associated with the wearer of the restraint device. The sensed parameter may be a physiological parameter of the wearer, or the sensed parameter may be a biological substance, a chemical substance, or a radioactive substance. The restraint device may further include a device communicating with the sensor and configured to store and/or transmit information related to the sensed parameter.

This application claims the priority of U.S. Provisional Patent Application Ser. No. 61/504,343, filed Jul. 5, 2011 (pending), the disclosure of which is incorporated by reference herein.

### TECHNICAL FIELD

The present invention relates generally to devices for limiting movement of an individual, and more particularly to a restraint device configured to provide information associated with a wearer of the restraint device.

### BACKGROUND

Various types of devices are known for restraining the free movement of an individual, or for restraining the free movement of a portion of the individual's body with respect to another portion of the individual's body. Restraint devices may be used, for example, to restrain the movement of a person who has been involved in an accident, or who has otherwise been injured, to isolate the injury and prevent further harm to the individual. Examples of such restraint devices include cervical collars, knee braces, spinal braces, and other similar devices.

In other situations, restraint devices may be used for law enforcement purposes, to restrict the free movement of persons who have been apprehended in connection with the commission of a crime, for example. Such restraint devices include, but are not limited to, handcuffs, leg shackles, waist chains, straightjackets, or electronic restraint devices.

In certain instances, it may be desirable to obtain information associated with the individual who has been restrained with a restraint device. For example, when restraint devices are used for medical purposes, it may be desirable to sense at least one physiological parameter associated with the wearer of the device, such as skin temperature, skin pH, heart rate, the level of blood-oxygen saturation, a virulent bacterial status, or any other physiological parameter, as may be desired. In other instances, it may be desirable to sense the presence of, or the exposure of the wearer to one or more substances, such as biological substances, chemical substances, or radioactive substances. For example, it may be desirable to determine whether the wearer of a restraint device has been exposed to, or has come in contact with, explosive materials, chemical weapons, radioactive materials, dangerous biological substances, illegal drugs, or other substances.

Conventionally, when it has been desired to detect a physiological parameter associated with the wearer, or to determine if the wearer has been exposed to various biological, chemical, or radioactive substances such as those described above, additional steps must be taken to detect the desired physiological parameter or substance. In many instances, a sample must be obtained from the wearer and delivered to an appropriate laboratory so that tests may be conducted on the obtained sample. As a result, significant delays in obtaining information related to the restrained individual, and communicating that information to appropriate persons occur. A need therefore exists for a restraint device that overcomes these and other deficiencies of the prior art.

### SUMMARY

The present invention overcomes the foregoing and other shortcomings and drawbacks of restraint devices heretofore known for use in various medical, law enforcement, or other applications. While the invention will be described in connection with certain embodiments, it will be understood that the invention is not limited to these embodiments. On the contrary, the invention includes all alternatives, modifications and equivalents as may be included within the spirit and scope of the present invention.

According to one aspect of the present invention, a restraint device includes at least a first restraint portion defining a space for receiving at least a first body portion of a wearer to restrain movement of the wearer. The restraint device may restrain movement of the at least one body portion relative to another body portion of the wearer, or the restraint device may restrain movement of the at least one body portion relative to a permitted space. The restraint device further includes at least one sensor on the first restraint portion. The sensor is configured to sense at least one parameter associated with the wearer of the restraint device.

In one aspect, the sensor may be configured to sense at least one physiological parameter of the wearer, such as skin temperature, skin pH, heart rate, the level of blood oxygen saturation, a virulent bacterial status, or any other physiological parameter, as may be desired. Alternatively, or additionally, the sensor may be configured to sense the presence of, or the exposure of the wearer to, at least one of a biological substance, a chemical substance, or a radioactive substance. The restraint device may further include a device communicating with the sensor and configured to store information related to the sensed parameter. The device may additionally or alternatively be configured to transmit to a remote location information related to the sensed parameter

These and other features, objects and advantages of the invention will become more readily apparent to those of ordinary skill in the art upon review of the following detailed description, taken in conjunction with the accompanying drawings.

### BRIEF DESCRIPTION OF THE FIGURES

FIG. 1 depicts an exemplary restraint device in accordance with the principles of the present disclosure, including a sensor for detecting a parameter associated with the wearer of the device.

FIG. 2 depicts another exemplary restraint device in accordance with the principles of the present disclosure, in the form of handcuffs.

FIG. 3 depicts a third embodiment of an exemplary restraint device in accordance with the principles of the present disclosure, in the form of an ankle bracelet.

FIG. 4 depicts a fourth exemplary embodiment of a restraint device in accordance with the principles of the present disclosure, in the form of a cervical collar.

### DETAILED DESCRIPTION

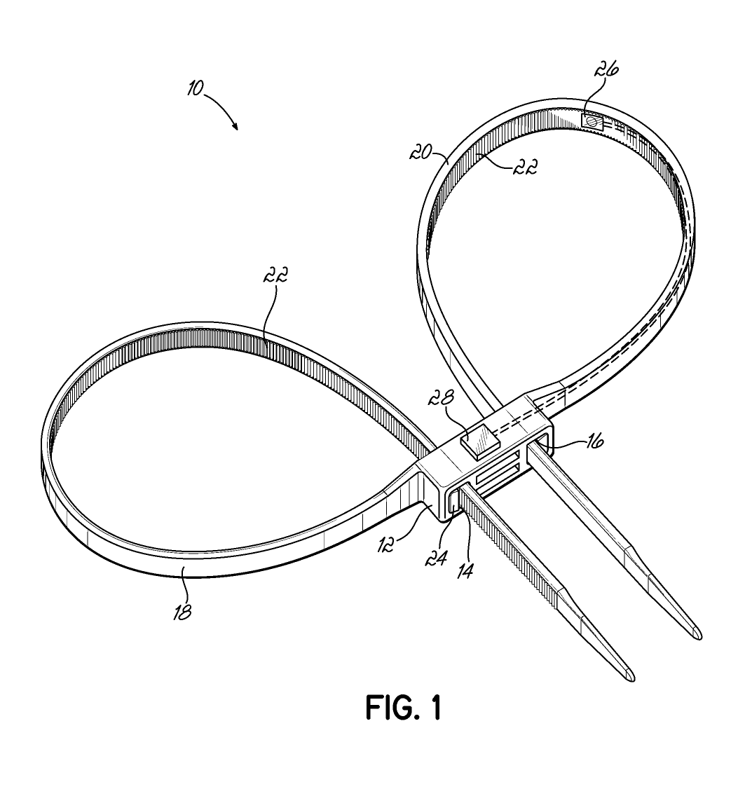

FIG. 1 depicts an exemplary restraint device 10 in accordance with the principles of the present invention, in the form of a double cuff restraint that can be applied to the wrist or ankles of a person to thereby restrain movement of the arms or legs of the person relative to one another. Such restraint devices 10 have been referred to as cable-tie or zip-tie type cuffs having a main body portion 12 with a pair of apertures 14, 16 through which respective first and second elongate straps 18, 20 may be directed to form loops or cuffs intended to surround the wrists or ankles of a person to be restrained. Each of the first and second elongate straps 18, 20 includes an integrally-formed gear rack having teeth 22 that can be engaged with a ratchet mechanism 24 disposed within the respective apertures 14, 16 to allow movement of the straps 18, 20 through the respective apertures 14, 16 in only one direction. Once the teeth 22 of the gear rack are engaged by the ratchet mechanism 24, the straps 18, 20 cannot be pulled back out of the apertures 14, 16. Therefore, once engaged, the straps 18, 20 can only be tightened.

The restraint device 10 further includes one or more sensors 26 for sensing at least one parameter associated with the wearer of the restraint device 10. In the embodiment shown, a sensor 26 is positioned on an inside portion of at least one of the straps 18, 20. In this configuration, the sensor 26 may contact the skin of a wearer, such that the sensor 26 is suitable to detect a physiological parameter associated with the wearer, such as, but not limited to, skin temperature, skin pH, heart rate, the level of blood-oxygen saturation, a virulent bacterial status, or any other physiological parameter of the wearer. In one embodiment, the sensor 26 may incorporate nanotechnology devices or structures to enable the sensor 26 to detect a virulent bacterial status of a wearer, such as, but not limited to Methicillin-resistant Staphylococcus aureus (MRSA), Beta Streptococcus, or other conditions of a wearer of the restraint device 10.

Additionally, or alternatively, the sensor 26 may be configured to sense the presence of, or the exposure of the wearer to, at least one of a biological substance, a chemical substance, or a radioactive substance. For example, a biological or chemical substance may be associated with an explosive material, a chemical weapon, or a dangerous biological substance. In other embodiments, the sensor 26 may alternatively, or additionally, be configured to detect the presence of or the exposure of the wearer to radioactive materials, illegal drugs, or other substances.

In accordance with another aspect, the restraint device 10 may be configured to indicate when a particular parameter associated with the wearer of the restraint device 10 has been detected. Indication of the detected parameter may be a visual indication, an audible indication, or a combination thereof. Alternatively, or in addition, the restraint device 10 may be configured to store and/or transmit information related to the sensed parameter. Indication of the detected parameter and/or storage or transmission of information related to a sensed parameter may be accomplished by the sensor 26, itself, or may be provided for by, or in combination with, other components of the restraint device 10.

In the embodiment shown, the restraint device 10 further includes a device 28 for receiving signals from the one or more sensors 26. The device 28 may be configured as a data storage device that is operable to store the information related to the one or more sensed parameters associated with the wearer, which stored data may be later obtained, communicated, or transmitted for use by a medical or law enforcement professional, for example. Alternatively, or in addition, the device 28 may be configured as a transmitter, operable to transmit information related to the one or more sensed parameters.

The information may be transmitted to a remote location, such as, but not limited to, a central data collection device (not shown), or it may be transmitted to personnel proximate the wearer of the restraint device 10. The stored or transmitted information may include the sensed parameters themselves, or may be related to the sensed parameters. As a non-limiting example, information related to a sensed parameter may be an alarm indicating that a particular condition exists (or has ceased to exist), or that a particular threshold of a sensed parameter has been traversed. The information may be stored or transmitted continuously or periodically, as may be desired. In an exemplary embodiment, information related to the sensed parameter of a wearer may be communicated to attendant personnel, such as medical or law enforcement professionals, whereby the attendant can take immediate action if needed. In other applications, the stored or transmitted information may be collected and analyzed with information from other sources, such as information from other restrained individuals, for research, law enforcement, or various other purposes.

While the restraint device 10 depicted in FIG. 1 is shown to include a single sensor 26 positioned for contact with the skin of a wearer of the restraint device 10, it will be appreciated that more than one sensor 26 may alternatively be used, and that the one or more sensors 26 may alternatively, or additionally, be placed on other portions of the restraint device 10, as may be desired. For example, when it is desired to sense a parameter that does not necessarily require contact between a sensor 26 and the skin of a wearer, the sensor 26 may be placed at other suitable positions on the restraint device 10. It will also be appreciated that one or more sensors 26 may be incorporated directly into the restraint device 10 during manufacture of the restraint device 10, or that one or more sensors 26 may be added to a pre-existing restraint device to obtain a restraint device 10 in accordance with the principles of the present disclosure.

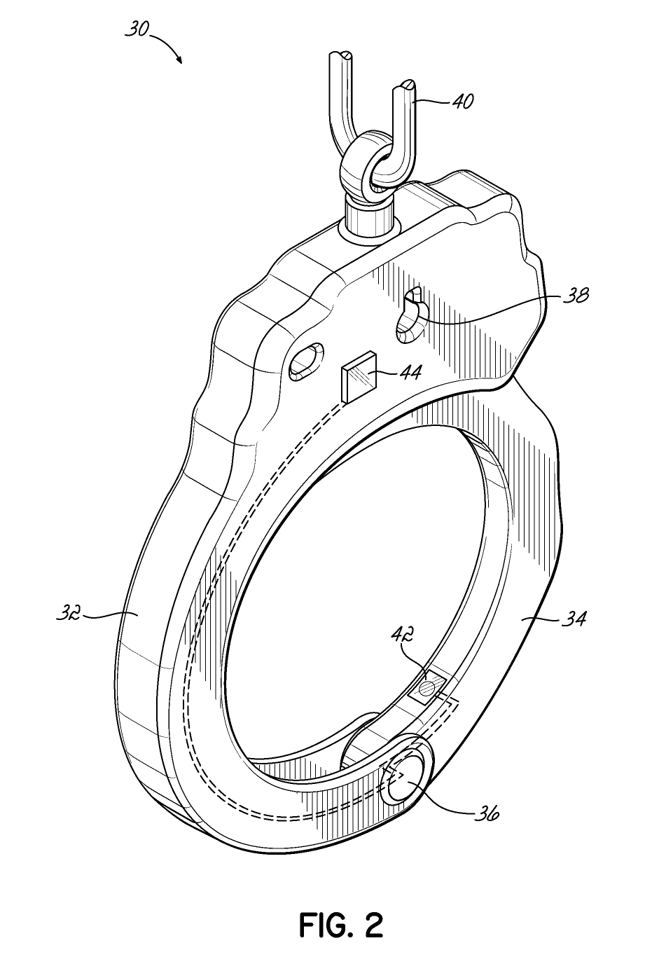

While the restraint device 10 depicted in FIG. 1 is generally a disposable type restraint, FIG. 2 depicts another exemplary embodiment of a restraint device in accordance with the principles of the present disclosure in the form of handcuffs 30, which may constitute a form of reusable restraint device. In the embodiment shown, the handcuffs 30 are of a conventional handcuff construction, including a cheek plate assembly 32 and an adjustable bow 34 pivotally coupled to the cheek plate assembly 32 by a pivot joint 36. While not specifically illustrated herein, the bow 34 includes a plurality of gear teeth that are engaged by a locking mechanism within the cheek plate assembly 32 and which are releasable by manipulation of an appropriate key inserted into a keyway 38. While the embodiment shown in FIG. 2 depicts only a single cuff, it will be appreciated that a pair of cuffs may be coupled together, such as by chain links 40, as known in the art.

With continued reference to FIG. 2, the handcuffs 30 include at least one sensor 42 positioned on the handcuffs 30 for sensing a desired parameter associated with the wearer of the handcuffs 30. In this embodiment, a sensor 42 is positioned on an inwardly facing portion of the bow 34 for contact with the skin of a wearer of the handcuffs 30. It will be appreciated, however, that more than one sensor 42 may be used with the handcuffs 30, and that the sensors 42 may be positioned on various other portions of the handcuffs 30, as may be desired. As discussed above, the sensor 42 may be configured to sense a physiological parameter associated with the wearer, such as a virulent bacterial status of the wearer, a skin temperature, a skin pH, a heart rate, or a blood-oxygen saturation of the wearer. Alternatively, or in addition, the sensor 42 may be configured to sense the presence of, or the exposure of the wearer to, at least one of a biological substance, a chemical substance, or a radioactive substance.

The handcuffs 30 may be further configured to indicate detection of the sensed parameter, such as by audio, visual, or other methods. Indication of a detected parameter may be accomplished by the sensor 42, itself, or indication may be provided for by, or in combination with, additional components of the handcuffs 30. In the embodiment shown, the handcuffs 30 include a device 44 for receiving signals from the one or more sensors 42. The device 44 may be configured as a data storage device operable to store information related to the sensed parameter of the wearer, or as a transmitter operable to transmit information related to the sensed parameter to a remote location, as discussed above.

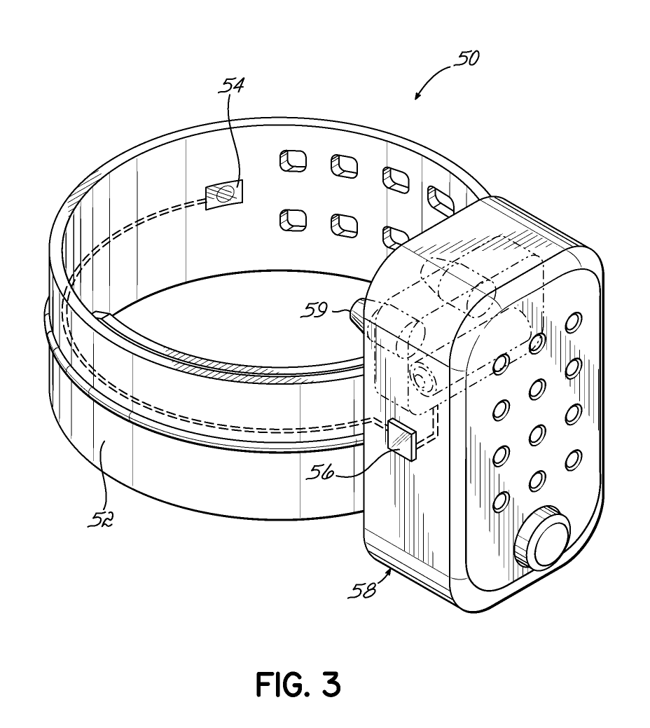

While the restraint devices 10, 30 depicted in FIGS. 1 and 2 illustrate embodiments configured to restrain movement of at least a first body portion of a wearer relative to a second body portion of the wearer, FIG. 3 illustrates another exemplary embodiment of a restraint device in accordance with the principles of the present disclosure wherein the device is configured to limit movement of at least a first body portion of the wearer with respect to a defined location or space. In this embodiment, the restraint device is in the form of a single cuff 50 that may be placed upon the ankle, wrist, or other suitable portion of an individual's body. To this end, the cuff 50 includes an elongate band 52 that may be placed around a portion of the wearer's body, such as an ankle. With the band 50 fitted securely to the wearer, the band 52 may be locked in place so that it cannot easily be removed by the wearer. The cuff 50 further includes one or more sensors 54 positioned on the cuff 50 to sense a desired parameter associated with the wearer of the cuff 50. In the embodiment shown, a sensor 54 is positioned on a portion of the band 52 for contact with the skin of the wearer. It will be appreciated, however, that the one or more sensors 54 may be positioned on various other portions of the cuff 50, as may be desired.

As discussed above, the sensor 54 may be configured to sense a physiological parameter associated with the wearer, such as a virulent bacterial status of the wearer, a skin temperature, a skin pH, a heart rate, or a blood-oxygen saturation of the wearer. Alternatively, or in addition, the sensor 54 may be configured to sense the presence of, or the exposure of the wearer to, at least one of a biological substance, a chemical substance, or a radioactive substance. The cuff 50 may be further configured to indicate detection of the sensed parameter, such as by audio, visual, or other methods. Indication of a detected parameter may be accomplished by the sensor 54, itself, or indication may be provided for by, or in combination with, additional components of the cuff 50.

In the embodiment shown, the cuff 50 further includes a device 56 for receiving signals from the one or more sensors 54. The device may be configured as a data storage device operable to store information related to the sensed parameter of the wearer or may be configured as a transmitter operable to transmit information related to the sensed parameter to a remote location as, discussed above. In this embodiment, the cuff 50 may be configured to transmit signals containing information, such as the location of the wearer of the cuff 50 and/or the one or more sensed parameters associated with the wearer of the cuff 50. If the wearer moves outside of a permitted space, signals may be transmitted to notify an appropriate monitoring authority. Alternatively, or in addition, the cuff 50 may be configured to emit an audible sound to indicate that the wearer of the cuff 50 has moved outside the permitted space.

In another embodiment, the cuff 50 may be configured to apply a disabling electric shock to incapacitate the wearer of the cuff 50 if an attempt is made to leave the permitted space. In such an embodiment, the cuff 50 may include a module 58 having electronic circuitry including a power source, one or more transformers, one or more capacitors, and other components capable of generating an electric charge and applying the electric charge to the wearer via one or more electrodes 59 positioned at an appropriate location on the cuff 50.

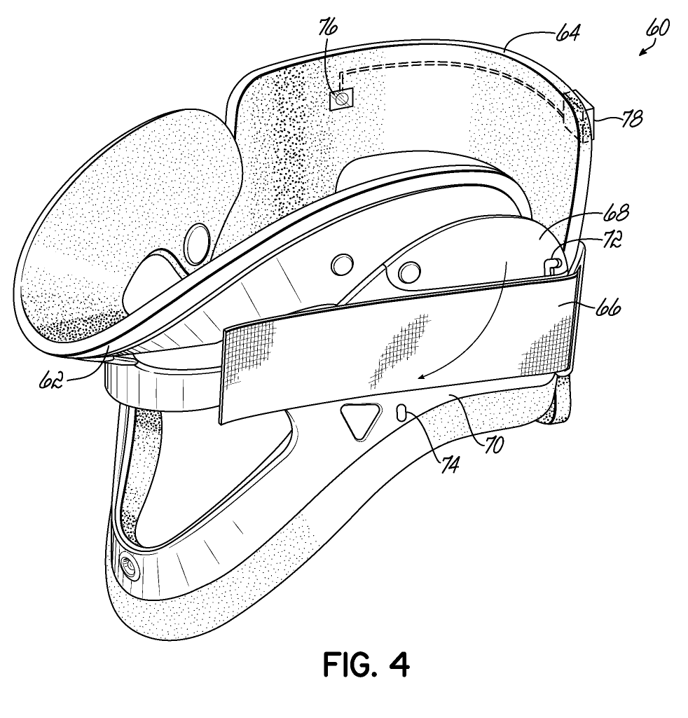

FIG. 4 depicts another exemplary restraint device in accordance with the principles of the present disclosure, in the form of a cervical collar 60. The cervical collar 60 depicted in FIG. 4 is similar to the cervical collar generally shown and described in U.S. Pat. No. 7,883,485, the disclosure of which is incorporated by reference herein in its entirety. The cervical collar 60 includes first and second support portions 62, 64 configured to immobilize, or at least brace the patient's head and neck region with respect to the rest of the wearer's body. In this embodiment, the first and second support portions 62, 64 may be arranged into a generally annular configuration and secured with a connector strap 66, such as a hook-and-loop type fastening strap system, for example. The cervical collar 60 further includes a pivoting lever arm 68 secured to a plastic piece 70 and pivotally moveable over the connector strap 66 after the cervical collar 60 has been fitted around the neck of the wearer. A slot or recess 72 formed into the pivoting lever arm 68 may be engaged with a corresponding latch 74 to contain the connector strap 66 and thereby prevent removal of the connector strap 66 from the first support portion 62 of the cervical collar 60 until the lever arm 68 is unlocked and pivoted out of the way, as generally described in U.S. Pat. No. 7,883,485.

The cervical collar 60 further includes one or more sensors 76 disposed on a portion of the collar 60 to sense one or more parameters associated with the wearer of the collar 60. In the embodiment shown, a sensor 76 is provided on an interior portion of the collar 60 at a location for contacting the skin of the wearer. It will be appreciated that more than one sensor 76 may alternatively be provided, and that such sensors 76 may alternatively, or additionally, be positioned on any suitable surface of the collar 60 to sense a desired a parameter. The one or more sensors 76 may be configured to sense a physiological parameter associated with the wearer, such as a virulent bacterial status of the wearer, a skin temperature, a skin pH, a heart rate, or a blood-oxygen saturation of the wearer. Alternatively, or in addition, the sensors 76 may be configured to sense the presence of, or the exposure of the wearer to, at least one of a biological substance, a chemical substance, or a radioactive substance.

The cervical collar 60 may be further configured to indicate detection of the sensed parameter, such as by audio, visual, or other methods. Indication of a detected parameter may be accomplished by the sensor 76, itself, or indication may be provided for by, or in combination with, additional components of the cervical collar 60. In the embodiment shown, the cervical collar 60 further includes a device 78 for receiving signals from the one or more sensors 76. The device 78 may be configured as a data storage device operable to store information related to the sensed parameter of the wearer or, alternatively or in addition, the device 78 may be configured as a transmitter operable to transmit information related to the sensed parameter to a remote location.

In use, a restraint device, such as, but not limited to, any of the restraint devices 10, 30, 50, 60 described herein, may be placed on at least a portion of an individual's body such that the restraint device 10, 30, 50, 60 restrains the movement of at least the portion of the individual's body. The restraint device 10, 30, 50, 60 may restrain movement of at least the portion of the individual's body with respect to another portion of the individual's body, or the restraint device 10, 30, 50, 60 may restrain movement with respect to a permitted space. The restraint device 10, 30, 50, 60 includes at least one sensor 26, 42, 54, 76 as described above, whereby the at least one sensor 26, 42, 54, 76 senses at least one parameter associated with the wearer. Once the parameter is sensed, the restraint device 10, 30, 50, 60 may be configured to indicate that the sensed parameter has been detected so that appropriate action may be taken. Information related to the sensed parameter may be stored or may be transmitted to appropriate personnel, such as medical or law enforcement personnel, which may be located remote from the wearer of the restraint device 10, 30, 50, 60.

In another aspect in accordance with the principles of the present invention, a restraint device 10, 30, 50, 60, such as those described above, may be configured to provide visual indication of the detection of a sensed parameter in dark or low-light environments. For example, the sensor 26, 42, 54, 76, the device 28, 44, 56, 78, or other components of the restraint device 10, 30, 50, 60 may be configured to illuminate upon the detection of a sensed parameter. Illumination may be provided by phosphorescent materials, electrical components, or any other suitable components or features capable of providing or facilitating visual indication in dark or low-light conditions. Moreover, the restraint devices themselves may be formed from or with phosphorescent or other luminous materials to facilitate visibility of the restraint device 10, 30, 50, 60 in dark or low-light conditions.

Within the context of this patent the purpose of a sensor is to monitor the environment surrounding a restrain device. This monitoring would include measuring properties both directly and indirectly of a number of sensors.

Examples of such sensors would include but not be limited to:

1. Signal transducers such as: photoelectric, photodielectric, photoluminescence, electroluminescence, and chemiluminescence, thermoelectric.

2. Transduction platforms such as: optical waveguide based transducers (optical fiber, interferometric optical transducers or surface plasmon resonance transducers).

3. Conductometric and capacitive transducers (nanostructured SnO₂ thin film exposed to O₂ gas at 400° C.).

4. Electrochemical transducers (electrochemical pH sensors).

5. Solid state transducers (p-n diodes or bipolar junction based transducers or Schottky diode based or MOS based).

6. Inorganic nanotechnology enabled sensors (gas sensing nanostructured thin films, phonons, nano-mechanical sensors, nano enabled optical sensors, nano enabled magnetically engineered spintronic sensors).

7. Organic nanotechnology enabled sensors such as proteins, antibodies, enzymes, etc. Nano-sensors based on nucleotides and DNA.

8. Acoustic wave transducers such as film bulk acoustic wave resonator, cantilever based transducers or inerdigitally launched surface acoustic wave devices (SAW).

While the present invention has been illustrated by the description of one or more exemplary embodiments, and while the embodiments have been described in considerable detail, they are not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and method and illustrative examples shown and described. Accordingly, departures may be made from such details without departing from the scope or spirit of the general inventive concept. What is claimed is:

1. A restraint device, comprising:
at least a first restraint portion defining a space for receiving at least a first body portion of a wearer and restraining movement of the wearer; and
at least one sensor on said first restraint portion, said sensor configured to sense at least one parameter associated with a wearer of the restraint device.

2. The restraint device of claim 1, wherein said first restraint portion limits motion of the first body portion with respect to a second body portion of the wearer.

3. The restraint device of claim 1, wherein said first restraint portion limits motion of the first body portion with respect to a defined location.

4. The restraint device of claim 1, wherein said sensor is configured to sense a physiological parameter associated with the wearer.

5. The restraint device of claim 4, wherein said sensor is positioned for contact with the skin of the wearer.

6. The restraint device of claim 4, wherein said sensor is configured to sense a virulent bacterial status of the wearer.

7. The restraint device of claim 4, wherein said sensor is configured to sense at least one of a skin temperature, a skin pH, a heart rate, or a blood oxygen saturation of the wearer.

8. The restraint device of claim 1, wherein said sensor is configured to sense the presence of, or exposure of the wearer to, at least one of a biological substance, a chemical substance, or a radioactive substance.

9. The restraint device of claim 8, wherein the biological or chemical substance is associated with at least one of an explosive material, a chemical weapon, or a dangerous biological substance.

10. The restraint device of claim 1, wherein said sensor comprises nanotechnology components adapted to sense the at least one parameter.

11. The restraint device of claim 1, further comprising an indicator for indicating detection of the at least one parameter.

12. The restraint device of claim 11, wherein said indicator is configured to illuminate upon detection of the at least one parameter.

13. The restraint device of claim 1, further comprising:
a device for receiving signals from said at least one sensor and adapted to store information related to the at least one sensed parameter.

14. The restraint device of claim 1, further comprising:
a device for communicating with said at least one sensor and adapted to transmit to a remote location information related to the at least one sensed parameter.

15. The restraint device of claim 1, further comprising:
a device for delivering an electrical charge to the wearer.

16. The restraint device of claim 1, wherein at least a portion of the restraint device is configured to illuminate to facilitate visibility in low-light conditions.

17. A method of sensing at least one parameter associated with an individual, comprising:
restraining the movement of at least a portion of the individual's body using a restraint device having at least one sensor; and
sensing the at least one parameter with the sensor.

18. The method of claim 17, wherein sensing the at least one parameter comprises sensing a physiological parameter associated with the wearer.

19. The method of claim 17, wherein sensing the at least one parameter comprises sensing the presence of, or exposure of the wearer to, at least one of a biological substance, a chemical substance, or a radioactive substance.

20. The method of claim 17, further comprising:
indicating detection of the at least one parameter.

21. The method of claim 17, further comprising:
storing information related to the at least one sensed parameter.

22. The method of claim 17, further comprising:
transmitting information related to the at least one sensed parameter to a remote location.
